(19) United States
(12) Patent Application Publication
(10) Pub. No.: US 2014/0052081 A1
(43) Pub. Date: Feb. 20, 2014

(54) DONEPEZIL TRANSDERMAL PATCH

(71) Applicants: Kuo-Hua Yang, Taoyuan city (TW); Chih-Sheng Yang, Taoyuan city (TW); Hsuen-Yau Lin, Taoyuan city (TW)

(72) Inventors: Kuo-Hua Yang, Taoyuan city (TW); Chih-Sheng Yang, Taoyuan city (TW); Hsuen-Yau Lin, Taoyuan city (TW)

(73) Assignee: TAIWAN BIOTECH CO. LTD., TAOYUAN CITY (TW)

(21) Appl. No.: 13/979,579

(22) PCT Filed: Jan. 12, 2012
(86) PCT No.: PCT/US12/21145

§ 371 Date: Nov. 5, 2013

(30) Foreign Application Priority Data
Jan. 12, 2011 (TW) 100101169

(51) Int. Cl. A61K 9/70; A61K 31/445

(52) U.S. Cl. CPC A61K 9/7061; A61K 31/445
USPC 604/307; 514/319; 424/443

(57) ABSTRACT

The present invention relates to a donepezil transdermal patch comprising a backing layer and a pressure sensitive adhesive matrix layer, wherein the pressure sensitive adhesive matrix layer comprises donepezil free base and an acrylic pressure sensitive adhesive agent selected from the group consisting of a copolymer of 2-ethylhexyl acrylate and vinyl acetate and a copolymer of 2-ethylhexyl acrylate, vinyl acetate, and hydroxyl-containing monomers, and wherein 2-ethylhexyl acrylate is present in an amount of about 65% to about 75%, vinyl acetate is present in an amount of about 25% to about 35%, and the hydroxyl-containing monomers are in an amount of about 0% to about 10%, based on the total weight of the copolymer.

### FIELD OF THE INVENTION

The present invention relates to a transdermal patch, and more particularly to a donepezil transdermal patch having excellent transdermal absorption properties and long-lasting drug effects.

### DESCRIPTION OF THE PRIOR ART

Dementia, especially Alzheimer's disease, is a disease that is more likely to be developed by elderly individuals and requires more medical care and social costs. Progress in medical technology leads to a surge in the elderly population. Therefore, the development of anti-Alzheimer's disease drugs is gradually being demanded.

Donepezil, (±)-2,3-dihydro-5,6-dimethoxy-2-[[1-(phenylmethyl)-4-piperidinyl]methyl]-1H-inden-1-one, is a piperidine compound having acetylcholinesterase inhibiting activity that is used to treat Alzheimer's disease. Currently, most Alzheimer's disease treatment take the form of tablets for oral administration. However, as most Alzheimer's disease patients are elderly individuals, it is hard for them to take the oral agents. Moreover, it is difficult for patients having symptoms of Alzheimer's disease to take the oral agents. In addition, Alzheimer's disease patients usually have the symptoms of memory loss and dementia, and usually forget or refuse to receive medical treatment. Therefore, a donepezil transdermal patch is therefore developed to solve the problems described above.

Generally, a transdermal patch consists of an adhesive matrix layer, a backing layer and a release liner, wherein the adhesive matrix layer comprises drug, adhesive and other additives. However, the stratum corneum layer has a very high fat solubility, skin permeability of drug is generally low and the stratum corneum layer of normal skin has a barrier function to prevent invasion of exogenous materials; therefore, when a conventional transdermal patch is used, in many cases a drug blended therein cannot be sufficiently absorbed transdermally. Therefore, to increase the transdermal absorption properties of a drug in a transdermal patch, various studies have been carried out.

U.S. 2008/0138399 A1 discloses a transdermal absorption patch comprising donepezil hydrochloride in high concentration, adhesive matrix and a (meth)acrylate copolymer having a carboxyl group. However, long-term use of donepezil hydrochloride in high concentration may cause the patients to have side effects such as nausea, vomiting, diarrhea, gastric juice increase, headache, insomnia, dizziness, fatigue, syncope, hot flushes, blood pressure changes, and muscle spasm.

U.S. 2010/0080842 A1 discloses a transdermal extended-delivery donepozil active agent composition comprising a donepozil active agent, a transdermal absorption enhancer, and a pressure sensitive adhesive comprised of carboxylic acid functionalities. Said composition is formulated to provide the donepozil active agent with a skin permeation rate of at least 1.5 μg/cm²/hr. However, the highest skin permeation rate of the donepozil active agent exemplified in the examples of U.S. 2010/0081842 only is only 3.4 μg/cm²/hr.

Therefore, we are still looking for a transdermal patch that is suitable to be used in treating Alzheimer's disease, and that has sufficient transdermal absorption properties and long-lasting drug effect.

### SUMMARY OF THE INVENTION

Accordingly, a primary objective of the present invention is to solve the problems described above, and to provide a donepezil transdermal patch having excellent trasdermal absorption properties to achieve a satisfying bioavailability.

Another objective of the present invention is to provide a donepezil transdermal patch having long-lasting drug effects, which can be effectively applied to a region on the skin of patients for at least three days.

The other objective of the present invention is to provide a method for treating Alzheimer's disease, which comprises applying the transdermal patch of the present invention to skin of patients.

### BRIEF DESCRIPTION OF THE DRAWINGS

FIG. 1 shows the correlation between the time and the skin permeation amounts of the donepezil transdermal patches of the examples of the present invention and of the comparative examples.

FIG. 2 shows the correlation between the time and the skin permeation amounts of the donepezil transdermal patches comprising different permeation enhancers of the present invention.

FIG. 3 shows the relationship of the skin permeation amounts of the donepezil transdermal patch of Example B1 of the present invention with time.

FIG. 4 shows the trial test results of the donepezil transdermal patch of the present invention on human subjects and the test results of the oral administration of donepezil on human subjects.

FIG. 5 shows the result of a simulated test involving the donepezil transdermal patch of the present invention and the oral administration of donepezil when the plasma concentration of multiple dosages thereof is in a steady state.

### DETAILS OF THE INVENTION

Hereinafter, preferred embodiments of the present invention are illustrated in detail.

The donepezil transdermal patch of the present invention comprises:

(a) a backing layer, and
(b) a pressure sensitive adhesive matrix layer comprising:
(i) donepezil free base, and
(ii) an acrylate pressure sensitive adhesive agent selected from the group consisting of a copolymer of 2-ethylhexyl acrylate and vinyl acetate and a copolymer of 2-ethylhexyl acrylate, vinyl acetate, and hydroxyl-containing monomers, wherein 2-ethylhexylacrylate is present in an amount of about 65% to about 75%, vinyl acetate is present in an amount of about 25% to about 35%, and the hydroxyl-containing monomers are present in an amount of about 0% to about 10%, based on the total weight of the copolymer.

In the present invention, the backing layer is not particularly limited and is well known by persons with ordinary skill in the art. In one embodiment of the present invention, the backing layer is prepared from polyester, nylon, polyvinyl chloride, polyethylene, polypropylene, ethylene-vinyl acetate copolymer, ionic polymer resins or other synthetic components.

The donepezil free base included in the transdermal patch of the present invention is well known to persons with ordinary skill in the art and can be prepared by any conventional methods. In the transdermal patch of the present invention, the donepezil free base is preferably in non-crystalline form. That is, the pressure sensitive adhesive matrix layer does not comprise any solid or undissolved donepezil free base.

In the present invention, the donepezil free base is present in an amount of preferably about 1% to about 15%, more preferably about 3% to about 12%, about 5% to about 12%, about 8% to about 12%, about 3% to about 5%, about 3% to about 8%, about 5% to about 8%, about 6% to about 11%, about 7% to about 12% or about 9% to about 12%, and most preferably about 10% based on the total weight of the pressure sensitive adhesive matrix layer.

In the transdermal patch of the present invention, the acrylic pressure sensitive adhesive agent included in the pressure sensitive adhesive matrix layer is selected from the group consisting of a copolymer of 2-ethylhexyl acrylate and vinyl acetate and a copolymer of 2-ethylhexyl acrylate, vinyl acetate, and hydroxyl-containing monomers, wherein 2-ethylhexylacrylate is present in an amount of about 65% to about 75% and preferably about 67% to 72%, vinyl acetate is present in an amount of about 25% to about 35% and preferably about 28% to about 30%, and the hydroxyl-containing monomers are in an amount of about 0% to about 10% and preferably about 5% to 8%, based on the total weight of the copolymer.

In a preferred embodiment of the present invention, the pressure sensitive adhesive agent is selected from the group consisting of Gelva®GMS 737 (where its weight is made up of about 72% of 2-ethylhexyl acrylate and about 28% of vinyl acetate, and was purchased from Cytec Industries, Inc.), Gelva®GMS 788 (where its weight is made up of about 70% of 2-ethylhexyl acrylate and about 30% of vinyl acetate, and was purchased from Cytec Industries, Inc.), and Duro-tak®2516 (where its weight is made up of about 67% of 2-ethylhexyl acrylate, about 28% of vinyl acetate, and about 5% of the hydroxyl-containing monomers, and was purchased from Henkel Corporation).

In one embodiment of the present invention, the hydroxyl-containing monomers are not particularly restricted; for example, but not limited to, hydroxymethyl acrylate, methyl hydroxymethyl acrylate, hydroxyethyl acrylate, hydroxyethyl methacrylate, hydroxypropyl acrylate, hydroxypropyl methacrylate, hydroxybutyl acrylate, hydroxybutyl methacrylate, hydroxypentyl acrylate, hydroxypentyl methacrylate, hydroxyhexyl acrylate, and hydroxyhexyl methacrylate.

In the present invention, the acrylic pressure sensitive adhesive agent is present in an amount of about 50% to about 99%, preferably about 50% to about 95%, about 50% to about 90%, about 50% to about 85%, about 50% to about 80%, about 60% to about 95%, about 60% to about 90%, about 60% to about 85%, about 60% to about 80%, about 70% to about 95%, about 70% to about 90%, about 70% to about 88%, about 80% to about 95% or about 80% to about 90%, more preferably about 85% to about 92.5%, based on the total weight of the pressure sensitive adhesive matrix layer.

In the donepezil transdermal patch of the present invention, the pressure sensitive adhesive matrix layer may additionally comprise a suitable skin permeation enhancer, which is, for example but not limited to, lauryl lactate, isopropyl myristate, oleic alcohol, or N-methylpyrrolidone.

According to the present invention, the permeation enhancer is present in an amount of about 1% to about 10%, preferably about 3% to about 8% or about 3% to about 6%, and more preferably about 5%, based on the total weight of the pressure sensitive adhesive matrix layer.

As long as not reducing the therapeutic effects of Alzheimer's disease, the donepezil transdermal patch of the present invention can additionally comprise, if needed, suitable plasticizers, stabilizers, or other additives.

In the present invention, the thickness of the pressure sensitive adhesive matrix layer is not particularly restricted, and is preferably about 40 micrometers to about 200 micrometers, more preferably about 80 micrometers to about 120 micrometers, and most preferably about 90 micrometers to about 100 micrometers.

The transdermal patch of the present invention preferably comprises a release liner. Suitable release liners are well known in this field, and are comprised of but not limited to those prepared from polymers, such as polyethylene, polypropylene, and polyesters, or comprised of paper sources, preferably of polyethylene terephthalate (PET). In one embodiment of the present invention, the release liner has a partition line, so that the release liner can be easily removed from the transdermal patch without contacting the pressure sensitive adhesive matrix layer by fingers, and then the transdermal patch can be applied onto the skin. The partition line can be a straight line or a curved line; for example but not limited to, a horizontal line, a vertical line, a wavy line, and a jagged line.

In the transdermal patch of the present invention, a backside treating agent, such as a silicone backside treating agent, a fluorine backside treating agent and wax, can be coated on the backing layer so that the patch can be rolled up and does not need any release liner.

In one embodiment of the present invention, the transdermal patch may comprise a water-proof moisture permeable film on the other side of the backing layer.

The donepezil transdermal patch of the present invention is suitable for the use of treating general dementia, such as Alzheimer's disease, and can also be used in other diseases, such as anti-cerebrovascular dementia or prevention of migraines.

The donepezil transdermal patch of the present invention can be applied to suitable sites on patient's body; for example but not limited to, breast, back, upper arm, thigh, and forehead. The patch can be applied to the patient for at least 3 to 5 days so as to obtain the best treatment effect.

The method for producing the donepezil transdermal patch of the present invention is not particularly restricted as long as it is a conventional method. One example of the method comprises the steps of dissolving a composition comprising donepezil free base and an acrylic pressure sensitive adhesive agent in a solvent such as toluene, hexane, and ethyl acetate; coating the solution on a release liner or a backing layer; removing the solvent by drying; and then binding the laminate coated with the solution to a backing layer or a release liner.

The embodiments given below are intended for demonstrating the present invention, not limiting the scope of the present invention. Any modifications and variations that can be easily made by those skilled in the art fall within the scope of the disclosure of the specification and the appended claims of the present invention.

### EXAMPLES

### Example 1

### Preparation of Donepezil Transdermal Patches

The donepezil transdermal patches in which the acrylic pressure sensitive adhesive matrix layer comprises the components shown in Tables 1 and 2 are to be prepared. First, the donepezil free base, the acrylic pressure sensitive adhesive agents, and the permeation enhancers (if used) are mixed in ethyl acetate at room temperature. Then, the solution is coated on a PET release liner to form a pressure sensitive adhesive matrix layer having a thickness of about 95 micrometers. Ethyl acetate is removed by drying. Thereafter, the liner is bound to a polyester backing layer, so as to produce a donepezil transdermal patch. In Tables 1 and 2, Samples A1 to A3 and B1 to B4 are the examples of the present invention while Samples C1 to C5 are the comparative examples.

TABLE 1

| | Sample | | | | | | |
|---|---|---|---|---|---|---|---|
| | A1 | A2 | A3 | C1 | C2 | C3 | C4 |
| | (wt | (wt | (wt | (wt | (wt | (wt | (wt |
| | %) | %) | %) | %) | %) | %) | %) |
| Donepezil free base | 7.5 | 7.5 | 7.5 | 7.5 | 7.5 | 7.5 | 7.5 |
| Gelva ®GMS 737 | 92.5 | | | | | | |
| Gelva ®GMS 788 | | 92.5 | | | | | |
| Duro-Tak ®2516 | | | 92.5 | | | | |
| Gelva ®GMS 2873 | | | | 92.5 | | | |
| Duro-Tak ®2054 | | | | | 92.5 | | |
| Duro-Tak ®2677 | | | | | | 92.5 | |
| Duro-Tak ®2052 | | | | | | | 92.5 |
| ethyl acetate | q.s. | q.s. | q.s. | q.s. | q.s. | q.s. | q.s. |

q.s.: sufficient amount

TABLE 2

| | Sample | | | | |
|---|---|---|---|---|---|
| | B1 | B2 | B3 | B4 | C5 |
| | (wt %) | (wt %) | (wt %) | (wt %) | (wt %) |
| Donepezil free base | 10.0 | 10.0 | 10.0 | 10.0 | 10.0 |
| Gelva ®GMS 737 | 85.0 | 85.0 | 85.0 | 85.0 | 90.0 |
| lauryl lactate | 5.0 | | | | |
| isopropyl myristate | | 5.0 | | | |
| oleic alcohol | | | 5.0 | | |
| N-methylpyrrolidone | | | | 5.0 | |
| ethyl acetate | q.s. | q.s. | q.s. | q.s. | q.s. |

q.s.: sufficient amount

### Example 2

### Skin Flux Test

### 1. Samples: the Donepezil Transdermal Patches A1 to A3, B1 to B4, and C1 to C5

### 2. Test Method and Conditions

(1) Skin Permeation Device
Skin is cut into several pieces to a predetermined mass size, i.e., 1 cm². The pieces of skin are then positioned with the side by side cell of a skin permeation device. The temperature within an external jacket of the side by side cell is kept at 32° C. After peeling off the release liner of the donepezil transdermal patch, the matrix layer pieces are adhesively secured onto the pieces of skin. 3.5 ml of 20% polyethylene glycol (PEG 400) is then added into the cell as a medium. At appropriate times, 0.5 ml of the medium is extracted for testing and then an additional 0.5 ml of the medium is added into the cell.
(2) Receptor medium: 20% of polyethylene glycol (PEG 400)
(3) Temperature: 32° C.
(4) Sampling time: the 24th, 48th, and 72nd hour
(5) Sample analysis: the samples were analyzed by using chromatography technique

### 3. Results

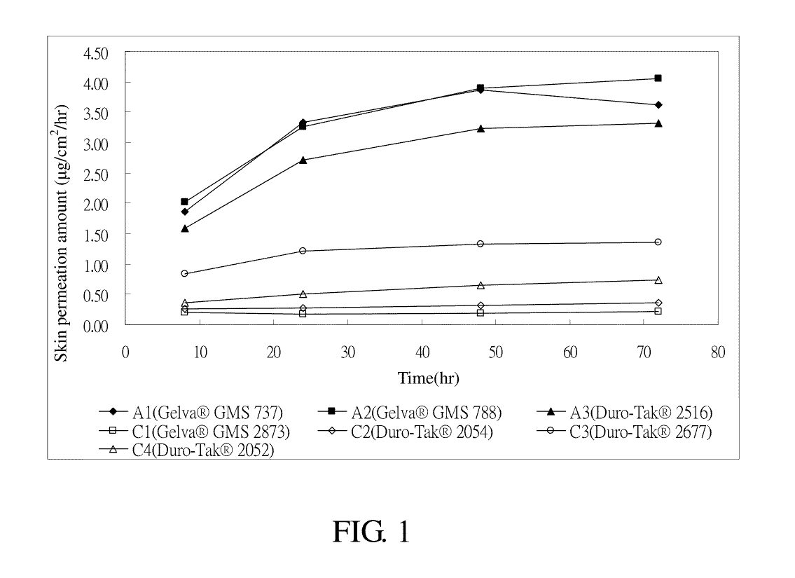

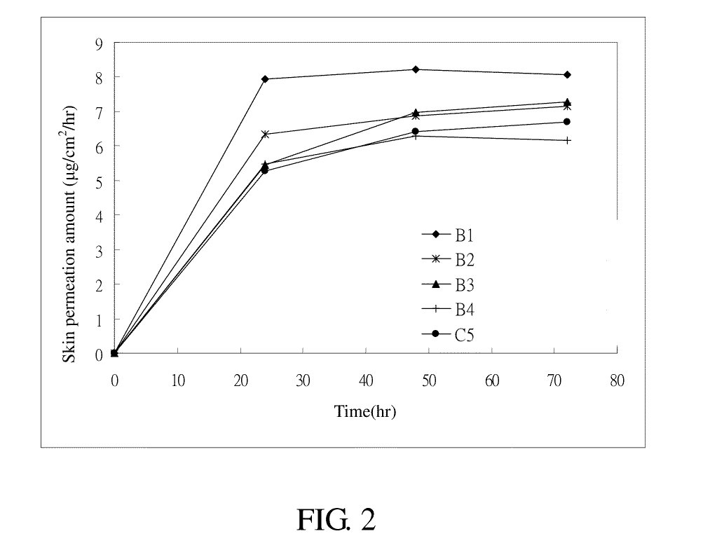

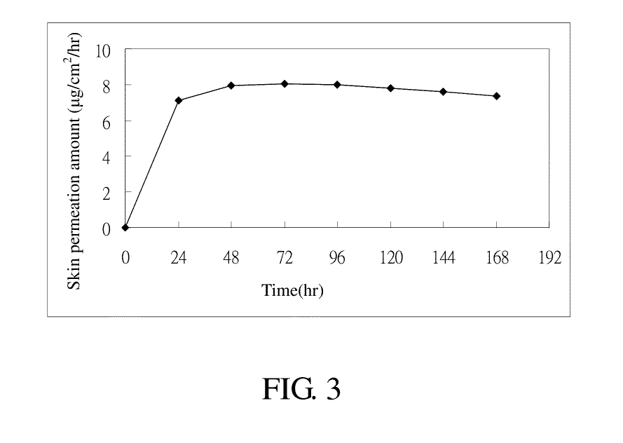

FIG. 1 shows the correlation between the time and the skin permeation amounts of the donepezil transdermal patches of the examples of the present invention and of the comparative examples. As shown in FIG. 1, after 72 hours, the skin permeation amounts of Samples A1, A2, and A3 are much higher than those of Samples C1, C2, C3, and C4. Among Samples A1 to A3, the skin permeation amounts of Sample A2 are the highest, Sample Al is the second, and Sample A3 is the last.
FIG. 2 shows the correlation between the time and the skin permeation amounts of the donepezil transdermal patches comprising different, or without, permeation enhancers of the present invention. As shown in FIG. 2, after 72 hours, the skin permeation amounts of Sample B1 are the highest, Samples B3 and B2 are the second, Sample C5 is the third, and Sample B4 is the last. In other words, Sample B1 has the best skin permeability than all of the other samples. The results reveal that the use of the permeation enhancers and the species thereof are relevant to the skin permeation amounts of the transdermal patch.
FIG. 3 shows the relationship of the skin permeation amounts of the donepezil transdermal patch of Example B1 of the present invention with time. As shown in

FIG. 3, the skin permeation amounts of the donepezil transdermal patch of the present invention reached an extreme level after approximately 48 hours, and the highest permeation rate is approximately 8.03 μg/cm2/hr. The transdermal patch can continuously and steadily provides drug activity for at least 3 days, and can even last for 7 days.
Table 3 shows the main components and the skin permeation results at a steady state of the donepezil transdermal patch of the present invention (Sample B1) and the donepezil transdermal patch of U.S. 2010/0080842 A1 (formulations of Sample 4 in Table 1 and Sample 4 in Table 3 of the specification (Samples C6 and C7)).

TABLE 3

| | Sample | | |
|---|---|---|---|
| | B1 | C6 | C7 |
| | (wt %) | (wt %) | (wt %) |
| Donepezil free base | 10.0 | 20.0 | 18.0 |
| Gelva ®GMS 737 | 85.0 | | |
| Durotak ®87-2852 | | 65.0 | |
| Durotak ®87-2196 | | | 72.0 |
| lauryl lactate | 5.0 | | |
| laureth4 | | 15.0 | 10.0 |
| skin permeation amounts at | about 7.96 | 3.3 | 3.4 |
| steady state | | | |
| (μg/cm²/hr) | | | |

As shown in Table 3, at steady state, the donepezil transdermal patch of the present invention has an excellent skin permeation capability, which is at least two-fold than that of the patch of U.S. 2010/0080842 A1.

### Example 3

### Pretest Results on Human Body

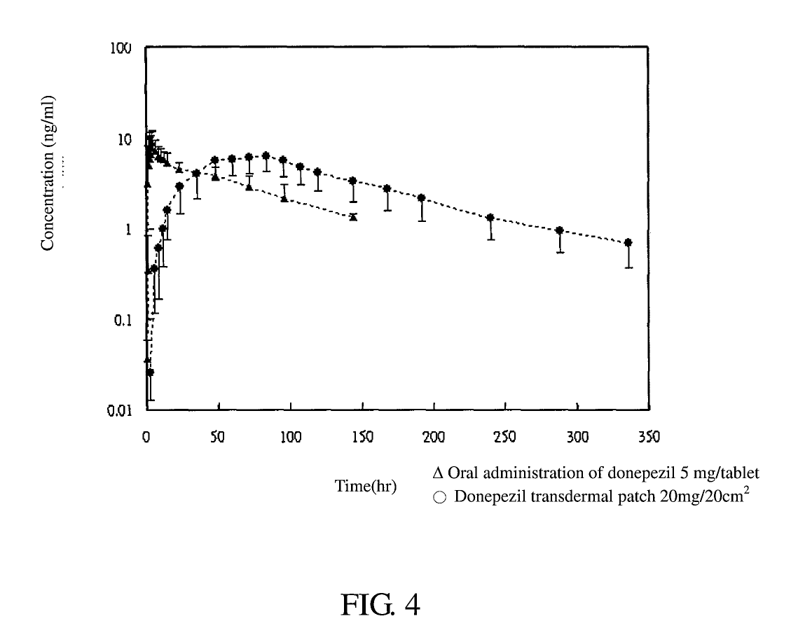

Four pieces of relatively the same size (approximately 20 mg/20 cm²) of Sample B1 in Table 2 were applied to the breast of four human subjects, and four tablets with 5 mg/tablet donepezil were orally administrated to another set of four human subjects. Then, the skin permeation rates of donepezil were tested by measuring the donepezil concentrations in blood samples that were drawn from the subjects at different time intervals. FIG. 4 shows the trial test results of the donepezil transdermal patch of the present invention on human subjects and the test results of the oral administration of donepezil on human subjects. As shown in FIG. 4, the highest concentration of donepezil in the blood samples of the subjects whom used the donepezil transdermal patch of the present invention (20 mg/20 cm²) and that of the subjects whom were orally administrated with donepezil tablets (5 mg/tablet) are similar.

### Example 4

### Simulated Test Results of Multi-Dosage on Human Subjects

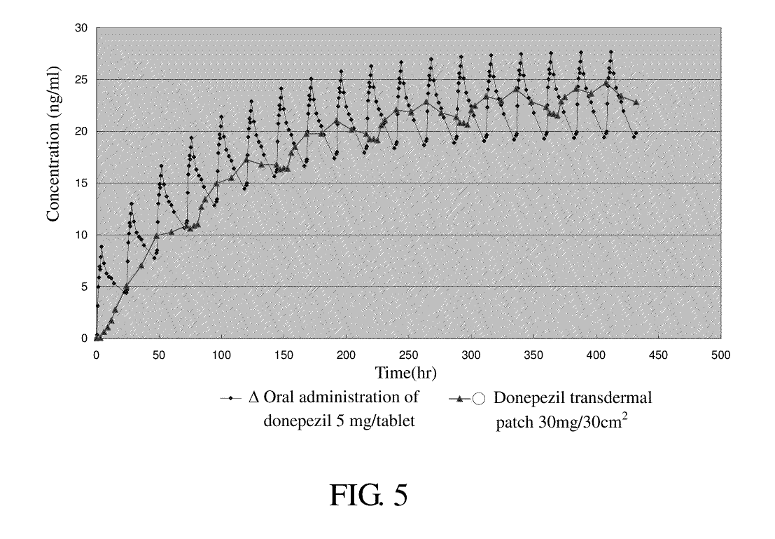

The simulated test of multi-dosage on human subjects is carried out by using the donepezil transdermal patch, Sample B 1. This simulation test is carried out on the basis of the human subject test of Example 3. FIG. 5 shows the simulated test results of the donepezil transdermal patch of the present invention (which is applied for 72 hours each time and is continuously replaced six times) and the oral administration of donepezil (which is administrated with 5 mg each time and the accumulation of the dosage is 18 times). As shown in FIG. 5, when the concentrations of donepezil in the blood plasma of the subjects whom using the patches and the oral tablets are at steady state, the average concentrations of donepezil in the blood plasma of the subjects using the donepezil transdermal patch of the present invention and using the donepezil oral tablet are similar. Based on these results, it can be estimated that the donepezil transdermal patch of the present invention has a similar treatment effect as to that of the oral administration of donepezil.

Given the above, the donepezil transdermal patch of the present invention is characterized by comprising a specific acrylic pressure sensitive adhesive agent and, if needed, using a specific skin permeation enhancer. The patch of the present invention can provide an excellent skin permeation amount and a better treatment effect without using a high concentration of the active ingredients. Moreover, solid or undissolved donepezil active agents are not generated in the pressure sensitive adhesive matrix layer so the patch does not need to use a solubilizing agent. The donepezil transdermal patch of the present invention provides long-term effects, sustains steadiness and lasting drug concentration in the blood, and can be applied continuously to a skin site of patients for at least 3 days. Given the above, when compared with conventional patches, the donepezil transdermal patch of the present invention have excellent transdermal absorption properties and long-lasting drug effects.

It will be apparent to those skilled in the art that various modifications and variations can be made to the structure of the present invention without departing from the scope or spirit of the present invention. In view of the foregoing, it is intended that the present invention covers modifications and variations of this invention, provided they fall within the scope of the following claims and their equivalents.

What is claimed is:

1. A donepezil transdermal patch, comprising:
(a) a backing layer, and
(b) a pressure sensitive adhesive matrix layer, comprising:
(i) donepezil free base,
(ii) an acrylate pressure sensitive adhesive agent selected from the group consisting of a copolymer of 2-ethylhexyl acrylate and vinyl acetate and a copolymer of 2-ethylhexyl acrylate, vinyl acetate, and hydroxyl-containing monomers, wherein 2-ethylhexyl acrylate is present in an amount of about 65% to about 75%, vinyl acetate is present in an amount of about 25% to about 35%, and the hydroxyl-containing monomers are present in an amount of about 0% to about 10%, based on the total weight of the copolymer;
(iii) a permeation enhancer selected from the group consisting of lauryl lactate, isopropyl myristate, and oleic alcohol;
wherein the donepezil free base is present in an amount of about 1% to about 15%, the acrylic pressure sensitive adhesive agent is present in an amount of about 50% to about 99%, and the permeation enhancer is present in an amount of about 1% to about 10%, based on the total weight of the pressure sensitive adhesive matrix layer.

2. (canceled)

3. The transdermal patch according to claim 1, wherein the donepezil free base is present in an amount of about 5% to about 12%, based on the total weight of the pressure sensitive adhesive matrix layer.

4. The transdermal patch according to claim 1, wherein the acrylic pressure sensitive adhesive is a copolymer of which its weight is about 72% of 2-ethylhexyl acrylate and about 28% of vinyl acetate.

5. The transdermal patch according to claim 1, wherein the acrylic pressure sensitive adhesive is a copolymer of which its weight is about 70% of 2-ethylhexyl acrylate and about 30% of vinyl acetate.

6. The transdermal patch according to claim 1, wherein the acrylic pressure sensitive adhesive is a copolymer of which its weight is about 67% of 2-ethylhexyl acrylate, about 28% of vinyl acetate, and about 5% of hydroxyl-containing monomers.

7. The transdermal patch according to claim 5, wherein the hydroxyl-containing monomers are selected from the group consisting of hydroxymethyl acrylate, methyl hydroxymethyl acrylate, hydroxyethyl acrylate, hydroxyethyl methacrylate, hydroxypropyl acrylate, hydroxypropyl methacrylate, hydroxybutyl acrylate, hydroxybutyl methacrylate, hydroxypentyl acrylate, hydroxypentyl methacrylate, hydroxyhexyl acrylate, and hydroxyhexyl methacrylate.

8. (canceled)

9. The transdermal patch according to claim 1, wherein the acrylic pressure sensitive adhesive agent is present in an amount of about 80% to about 95%, based on the total weight of the pressure sensitive adhesive matrix layer.

10. (canceled)

11. (canceled)

12. (canceled)

13. The transdermal patch according to claim 1, wherein the permeation enhancer is present in an amount of about 3% to about 6%, based on the total weight of the pressure sensitive adhesive matrix layer.

14. The transdermal patch according to claim 1 further comprising a release liner.

15. The transdermal patch according to claim 9, wherein the release liner has a partition line.

16. The transdermal patch according to claim 9, wherein the partition line is a straight line or a curved line.

17. A method for treating Alzheimer's disease, which comprises applying the transdermal patch of claim 1 to the skin of patients.
